(19) United States
(12) Patent Application Publication
(10) Pub. No.: US 2010/0100115 A1
(43) Pub. Date: Apr. 22, 2010

(54) FOAM FILLED INTRAGASTRIC BALLOON FOR TREATING OBESITY

(75) Inventors: Maximiliano Soetermans, Pinnacle, NC (US); Brian K. Rucker, King, NC (US); Victor D. Clark, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/254,626

(22) Filed: Oct. 20, 2008

(51) Int. Cl. A61M 29/02

(52) U.S. Cl. 606/192

(57) ABSTRACT

An apparatus and method comprising one or more intragastric balloons comprising a foam material disposed within the gastric lumen of a mammal. When the foam material is disposed within the one or more balloons, the one or more intragastric balloons are configured to prevent the intragastric device from passing through the mammal's pylorus. The one or more intragastric balloons are loaded onto a delivery tube in a partially compacted first configuration and delivered through an overtube. The overtube includes a proximal end, a distal end and a lumen configured to receive the one or more intragastric balloons in the first configuration for delivery into the gastric lumen wherein the one or more intragastric balloons are expanded to a second configuration upon delivery of the foam material. The foam material is delivered through an inflation tube attached to an opening of the one or more intragastric balloons.

### TECHNICAL FIELD

This invention relates to medical devices, and more particularly to obesity treatment devices that can be placed in the stomach of a patient to reduce the size of the stomach reservoir or to place pressure on the inside surface of the stomach.

### BACKGROUND OF THE INVENTION

It is well known that obesity is a very difficult condition to treat. Methods of treatment are varied, and include drugs, behavior therapy, and physical exercise, or often a combinational approach involving two or more of these methods. Unfortunately, results are seldom long term, with many patients eventually returning to their original weight over time. For that reason, obesity, particularly morbid obesity, is often considered an incurable condition. More invasive approaches have been available which have yielded good results in many patients. These include surgical options such as bypass operations or gastroplasty. However, these procedures carry high risks and are therefore not appropriate for most patients.

In the early 1980s, physicians began to experiment with the placement of intragastric balloons to reduce the size of the stomach reservoir, and consequently its capacity for food. Once deployed in the stomach, the balloon helps to trigger a sensation of fullness and a decreased feeling of hunger. These balloons are typically cylindrical or pear-shaped, generally range in size from 200-500 ml or more, are made of an elastomer such as silicone, polyurethane, or latex, and are filled with air, water, or saline. While some studies demonstrated modest weight loss, the effects of these balloons often diminished after three or four weeks, possibly due to the gradual distension of the stomach or the fact that the body adjusted to the presence of the balloon. Other balloons include a tube exiting the nasal passage that allows the balloon to be periodically deflated and re-insufflated to better simulate normal food intake. However, the disadvantages of having an inflation tube exiting the nose are obvious.

The experience with balloons as a method of treating obesity has provided uncertain results, and has been frequently disappointing. Some trials failed to show significant weight loss over a placebo, or were ineffective unless the balloon placement procedure was combined with a low-calorie diet. Complications have also been observed, such as gastric ulcers, especially with use of fluid-filled balloons, and small bowel obstructions caused by deflated balloons. In addition, there have been documented instances of the balloon blocking off or lodging in the opening to the duodenum, wherein the balloon may act like a ball valve to prevent the stomach contents from emptying into the intestines.

Unrelated to the above-discussed methods for treating obesity, it has been observed that the ingestion of certain indigestible matter, such as fibers, hair, fuzzy materials, etc., can collect in the stomach over time, and eventually form a mass called a bezoar. In some patients, particularly children and the mentally handicapped, bezoars often result from the ingestion of plastic or synthetic materials. In many cases, bezoars can cause indigestion, stomach upset, or vomiting, especially if allowed to grow sufficiently large. It has also been documented that certain individuals having bezoars are subject to weight loss, presumably due to the decrease in the size of the stomach reservoir. Although bezoars may be removed endoscopically, especially in conjunction with a device known as a bezotome or bezotriptor, they, particularly larger ones, often require surgery.

What is needed is an intragastric balloon that provides the potential weight loss benefits of a bezoar without the associated complications, and which overcomes some of the disadvantages of the fluid filled balloons described above. Ideally, such a device should be well-tolerated by the patient, effective over a long period of time, sizable for individual anatomies, and easy to place and retrieve. The device will also provide the benefit of short-term weight loss thereby preparing the patient to safely undergo subsequent medical procedures involving surgery.

### SUMMARY OF THE INVENTION

In one aspect of the invention, the obesity treatment apparatus comprises one or more intragastric balloons disposed within the gastric lumen of a mammal comprising a foam material disposed within the one or more intragastric balloons. The foam material includes one or more foam precursors which are configured to polymerize, foam, and cure upon introduction of the foam material into the one or more deployed intragastric balloons. When the foam material is disposed within the one or more intragastric balloons, the one or more intragastric balloons are configured to prevent the intragastric device from passing through the mammal's pylorus. The intragastric device also comprises an inflation tube attached to an opening of the one or more intragastric balloons for delivering foam material into the one or more intragastric balloons.

In another aspect of the invention, the obesity treatment apparatus comprises one or more intragastric balloons disposed within the gastric lumen of a mammal comprising a foam material and a plurality of intragastric members disposed within the one or more intragastric balloons. The intragastric members are introduced individually or in combination with the foam material depending on the diameter, design and the predetermined volume of the intragastric balloons. Irrespective of whether the obesity treatment apparatus includes only foam material, or a plurality of intragastric members in combination with the foam material, the principal requirement is that once disposed within the one or more intragastric balloons, the one or more intragastric balloons attain a shape and size that prevents the one or more intragastric balloons from passing through or lodging in the pyloric sphincter.

In another aspect of the invention, the obesity treatment apparatus comprises one or more intragastric balloons comprising a foam material disposed within a reinforcement member of the intragastric balloon. The reinforcement member comprises expandable ribs which expand from a first configuration to a second configuration upon receiving the foam material. The reinforcement member can comprise nitinol or similar material.

In another aspect of the invention, the obesity treatment apparatus includes a delivery system to deliver the one or more intragastric balloons comprising a foam material within the gastric lumen. In one embodiment, one or more intragastric balloons are mounted onto a delivery tube and secured with a releasing mechanism, such as a nylon thread, extending through the passageway of the delivery tube. A metal wire or loop is then withdrawn, severing the thread(s) and releasing the intragastric balloon(s) into the gastric lumen. The one or more intragastric balloons are then secured with a device such as a stopper pushed by an introduced metal tube or similar device. The foam material may be introduced from a pressurized canister and through a dedicated lumen of a catheter.

Other delivery systems of the present invention involve constraining the one or more intragastric balloons into a delivery tube, then releasing the one or more intragastric balloons within the gastric lumen and subsequently delivering foam material into the one or more intragastric balloons. Delivery of the one or more intragastric balloons can include pushing the one or more intragastric balloons through a passageway of the delivery tube, typically by use of a pusher member within the passageway of the delivery tube. Other methods include constraining the intragastric balloon(s) with a splittable or dissolvable film or sheath that allows the one or more intragastric balloons to be deployed in a compact configuration, then the one or more intragastric balloons are allowed to expand when the outer wrapping or sheath is split by the operator, or when the outer wrapping or sheath is allowed to dissolve away over time in the stomach. The dissolvable film or sheath of the one or more intragastric balloons comprises a material selected from the group consisting of cellulose, gelatin and glycerin.

In still yet another aspect of the invention, the one or more intragastric balloons can be precoupled together with a coupling mechanism, such as a nylon fishing line, prior to or after introduction into the gastric lumen. Because the volume of the grouping in the stomach increases over time due to mucous accumulation or other factors, a single device having the overall size of the grouping (e.g., two or more intragastric balloons grouped together) may not be readily removed. However, by severing the line comprising the coupling mechanism, the individual intragastric balloons of the grouping can be removed one at a time by using an endoscope and retrieval device.

In still yet another aspect of the invention, the obesity treatment apparatus can comprise one or more intragastric balloons made of a digestive-resistant material loaded onto a delivery tube in a partially compacted first configuration, wherein the assembly is delivered through an overtube. The overtube includes a proximal end, a distal end, and a lumen configured to receive the one or more intragastric balloons in the first configuration for delivery into the gastric lumen wherein the digestive-resistant material of the one or more intragastric balloons are expanded to a second configuration upon delivery of foam material through an inflation tube.

In still yet another aspect of the present invention, the obesity treatment apparatus can comprise one or more intragastric balloons deployed into the gastric lumen and comprising foam material and a plurality of intragastric members disposed within the foam material of the one or more intragastric balloons. The intragastric members can be disposed within the foam material of the one or more intragastric balloons separately or together to displace volume within the gastric lumen. The one or more intragastric balloons are then secured by pushing a stopper or similar device into the opening of the one or more intragastric balloons. Additionally, the intragastric members can be disposed within the foam material utilizing an elastic band attached to the opening of the one or more intragastric balloons which is inserted over an overtube wherein the remainder of the one or more intragastric balloons are inverted into the lumen of the overtube. The intragastric members are subsequently pushed into the one or more balloons and disposed within any foam material until the intragastric balloon is filled to a predetermined volume. A coaxial outer tube or similar device can be utilized to remove the elastic band from the overtube and thereby secure the one or more intragastric balloons with the elastic band.

The intragastric balloons may be removed by rupturing the one or more intragastric balloons resulting in the foam material passing through the gastrointestinal tract of the patient. Alternatively, the intragastric balloons can be removed by rupturing the one or more intragastric balloons and utilizing an overtube to suction the foam material and other existing matter from the one or more intragastric balloons and subsequently removing the one or more intragastric balloons through the overtube or endoscope with forceps or similar device. Further, the foam material can include a color coding to allow the foam material to be easily identified if the one or more balloons are prematurely ruptured.

In still yet another aspect of the invention, a method of treatment of obesity in mammals can comprise the steps of positioning one or more intragastric balloons within the gastric lumen of a mammal and delivering a foam material into the one or more intragastric balloons, wherein the one or more intragastric balloons are expanded from a first configuration to a second configuration upon delivery of the foam material into the one or more intragastric balloons. The second configuration is sufficiently large to prevent the one or more intragastric balloons from passing through the mammal's pylorus. The method also comprises the additional step of advancing the foam material through a lumen of an inflation tube attached to the one or more intragastric balloons within the gastric lumen.

In still yet another aspect of the invention, a method of treatment of obesity in mammals can comprise the step of positioning a delivery tube comprising the one or more intragastric balloons within a lumen of an overtube. The method further comprises the step of advancing an intragastric member through the lumen of the overtube and disposing the intragastric member within the foam material of the intragastric balloon. In addition, the method further comprises the step of securing a stopper to an opening of the one or more intragastric balloons within the gastric lumen.

These and other advantages, as well as the invention itself, will become apparent in the details of construction and operation as more fully described below. Moreover, it should be appreciated that several aspects of the invention can be used with other types of intragastric devices or procedures used for the treatment of obesity.

### BRIEF DESCRIPTION OF SEVERAL VIEWS OF THE DRAWINGS

Several embodiments of the present invention will now be described by way of example with reference to the accompanying drawings, in which:

FIG. 1 depicts a pictorial view of an embodiment of an intragastric balloon of the present invention;

FIG. 2 depicts a pictorial view of the embodiment of the intragastric balloon of FIG. 1 of the present invention, wherein the intragastric balloon is attached to an inflation tube;

FIG. 3 depicts a pictorial view of the embodiment of the intragastric balloon of FIG. 1 of the present invention upon delivery into the gastric lumen;

FIG. 4 depicts a pictorial view of another embodiment of an intragastric balloon of the present invention;

FIG. 5 depicts a pictorial view of the embodiment of the intragastric balloon of FIG. 4 of the present invention, wherein the intragastric balloon is attached to an inflation tube;

FIG. 6 depicts a pictorial view of the embodiment of the intragastric balloon of FIG. 4 of the present invention upon delivery into the gastric lumen;

FIG. 7 depicts a pictorial view of yet another embodiment of an intragastric balloon of the present invention;

FIG. 8 depicts a pictorial view of the embodiment of the intragastric balloon of FIG. 7 of the present invention, wherein the intragastric balloon is attached to an inflation tube;

FIG. 9 depicts a pictorial view of the embodiment of the intragastric balloon of FIG. 7 of the present invention upon delivery into the gastric lumen;

FIG. 10 depicts a pictorial view of another embodiment of a plurality of intragastric balloons of the present invention;

FIG. 11 depicts a pictorial view of yet another embodiment of an intragastric balloon of the present invention;

FIG. 12 depicts a pictorial view of the embodiment of the intragastric balloon of FIG. 11 of the present invention, wherein the intragastric balloon is attached to an inflation tube;

FIG. 13 depicts a pictorial view of the embodiment of the intragastric balloon of FIG. 1 of the present invention upon delivery into the gastric lumen;

FIG. 14 depicts a pictorial view of an embodiment of a delivery system of the present invention utilized to deliver an intragastric balloon into the gastric lumen;

FIG. 15 depicts a pictorial view of an embodiment of an intragastric balloon of the present invention, wherein the intragastric balloon is attached to an inflation tube in a first configuration;

FIG. 16 depicts a pictorial view of an embodiment of a delivery system of the present invention wherein the intragastric balloon of FIG. 15 is loaded into a delivery tube for delivery into the gastric lumen;

FIG. 17 depicts a pictorial view of an embodiment of a delivery system wherein the intragastric balloon of FIG. 15 is loaded onto a delivery tube for delivery into the gastric lumen;

FIG. 18 depicts a pictorial view of yet another embodiment of a delivery system wherein a plurality of intragastric balloons are secured with a coupling mechanism upon delivery into the gastric lumen;

FIG. 19 depicts a delivery system for introducing 2-component foam materials into an intragastric balloon;

FIG. 20 is a cross-sectional view of the catheter showing two lumens; and

FIG. 21 is a schematic of a canister containing precursor materials for production of a foam.

### DETAILED DESCRIPTION OF THE INVENTION

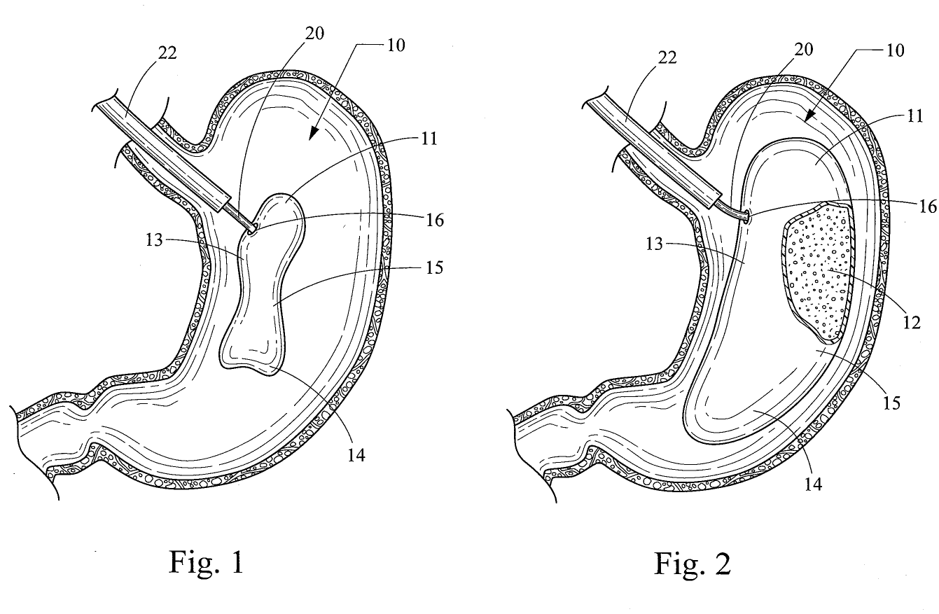

The obesity treatment apparatus 10 depicted in FIGS. 1-18 of the present invention comprises one or more intragastric balloons, each comprising a foam material sized and configured such that the foam material can be delivered into the one or more intragastric balloons placed into the gastric lumen of a mammalian patient and reside therein, and being generally unable to pass through the pylorus while remaining within the one or more intragastric balloons. As used herein, the term foam material is intended to refer to a material used to inflate the intragastric balloon and that is generally not subject to the degradative effects of stomach acid and enzymes, or the general environment found within the gastric system over an extended period of time, therefore allowing the device to remain intact for the intended life of the device. However, this does not necessarily mean that the foam material cannot be degraded over time. One skilled in medical arts and gastrological devices would readily appreciate the range of materials that would be suitable for use as foam material within the one or more intragastric balloons.

FIG. 1-3 depicts an embodiment of the obesity treatment apparatus 10 comprising a single intragastric balloon 11 comprising foam material 12 wherein the intragastric balloon 11 comprises a preformed expandable digestive-resistant material having a non spherical or elliptical shape. The intragastric balloon 11 includes a proximal end 13, a distal end 14 and a main body 15, wherein the proximal end 13 provides an opening 16 to receive an inflation tube 20.

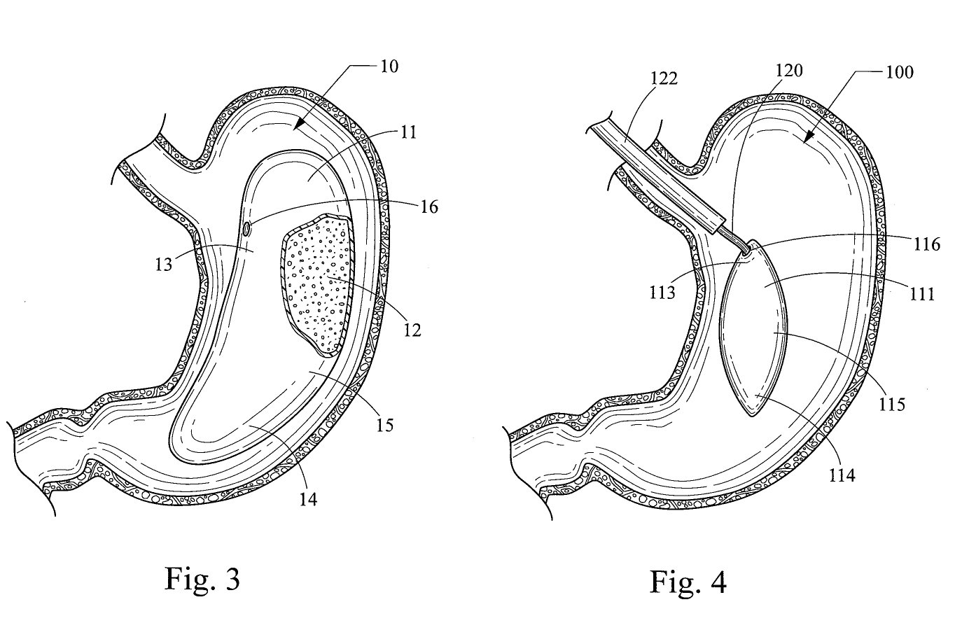

The intragastric balloon 11 is delivered into the gastric lumen of a mammal in a first configuration, wherein the first configuration is configured to permit introduction of the intragastric balloon 11 into the gastric lumen via the esophagus (see FIG. 1). The inflation tube 20 is passed through an outer tube 22 and attached to the intragastric balloon 11 to insert foam material 12 into the main body 15 of the intragastric balloon 11 (see FIG. 2). Upon receiving foam material 12, the intragastric balloon 11 is expanded from the first configuration to a second configuration, wherein the intragastric balloon 11 is sufficiently large to prevent the intragastric balloon 11 from passing through the mammal's pylorus (see FIG. 3). The expanded intragastric balloon 11 comprising foam material 12 engages the wall of the gastric lumen and reduces the volume of the gastric lumen thereby providing a feeling of fullness.

The intragastric balloon 11 may be inflated with foam material 12 to a volume ranging between about 50 percent and 100 percent of the maximum volume of the intragastric balloon 11, and may also vary depending on the size of the intragastric balloon 11 and the size of the gastric lumen of the particular patient. Once the foam material 12 has been delivered to the interior volume of the intragastric balloon 11, the inflation tube 20 can be removed from opening 16, as shown in FIG. 3. The opening 16 of the intragastric balloon 11 provides a self-sealing valve for securing foam material 12 within the main body 15 of the intragastric balloon 11. Alternatively, the opening 16 may comprise a stopper, suture, plug or similar mechanism to seal the opening 16 and prevent leakage of foam material 12 from the intragastric balloon 11 and into the gastric lumen of a mammal and still fall within the scope of the present invention.

FIG. 4-6 depicts another embodiment of the obesity treatment apparatus 100 comprising a single intragastric balloon 111 comprising foam material 112. The intragastric balloon 111 comprises a preformed expandable digestive-resistant material having a substantially spherical shape. The intragastric balloon 111 includes a proximal end 113, a distal end 114 and a main body 115, wherein the proximal end 113 provides an opening 116 to receive an inflation tube 120.

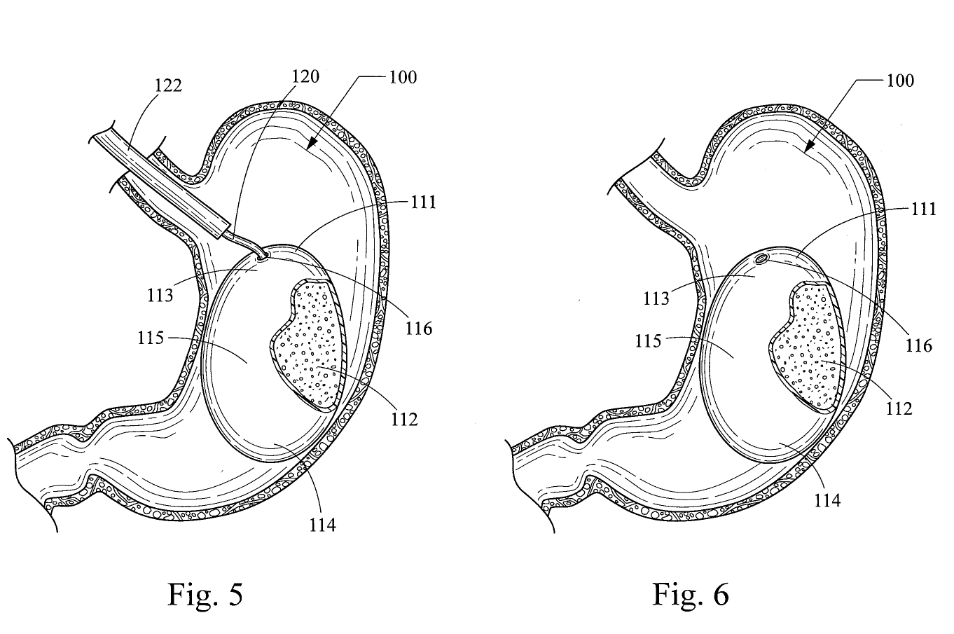

The intragastric balloon 111 is delivered into the gastric lumen of a mammal in a first configuration, wherein the first configuration is configured to permit introduction of the intragastric balloon 11 (see FIG. 4). In particular, the intragastric balloon 111 is delivered to the gastric lumen by passing it through outer tube 122. The inflation tube 120 is likewise passed through the outer tube 122 and attached to the intragastric balloon 111 to insert foam material 112 into the main body 115 of the intragastric balloon 111 (see FIG. 5). Upon receiving foam material 112, the intragastric balloon 111 is expanded from the first configuration to a second configuration, wherein the intragastric balloon 111 is sufficiently large to prevent the intragastric balloon 111 from passing through the mammal's pylorus (see FIG. 6). The inflation tube 120 and the outer tube 122 are then detached from the intragastric balloon 111 and removed from the patient. The expanded intragastric balloon 111 comprising foam material 112 engages the wall of the gastric lumen and reduces the volume of the gastric lumen thereby providing a feeling of fullness.

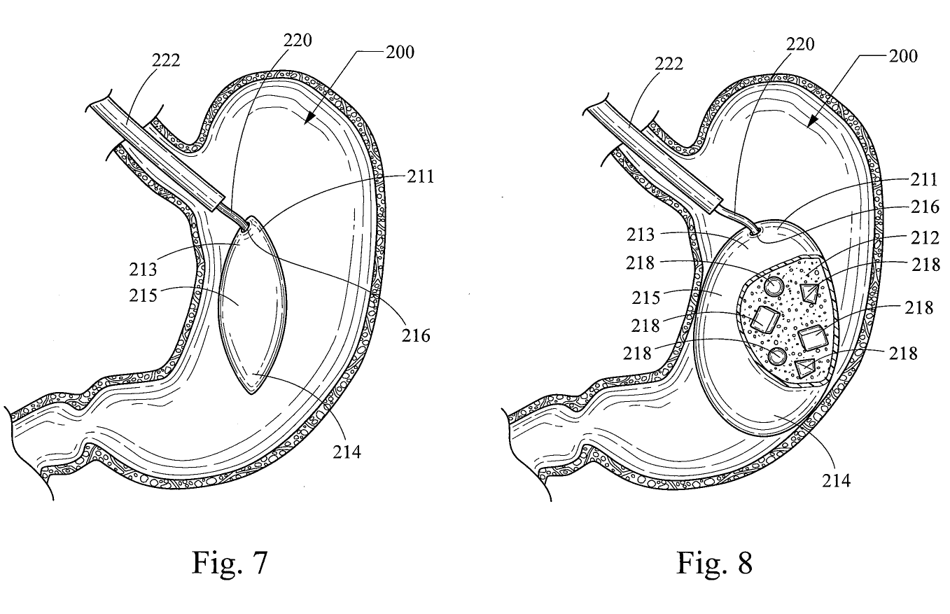

FIGS. 7-9 depicts yet another embodiment of the obesity treatment apparatus 200 comprising a single intragastric balloon 211 comprising foam material 212 and a plurality of intragastric members 218 disposed within the foam material 212. The intragastric balloon 211 comprises a preformed expandable digestive-resistant material having a substantially spherical shape. The intragastric balloon 211 includes a proximal end 213, a distal end 214 and a main body 215, wherein the proximal end 213 provides an opening 216 to receive an inflation tube 220.

The intragastric balloon 211 is delivered into the gastric lumen of a mammal in a first configuration via outer tube 222, wherein the first configuration is configured to permit introduction of the intragastric balloon 211 through outer tube 222 and into the gastric lumen (see FIG. 7). The inflation tube 220 is likewise passed through an outer tube 222 and is attached to the intragastric balloon 211 so as to deliver the foam material 212 into the main body 215 of the intragastric balloon 211 (see FIG. 8). The inflation tube 220 or similar device can also be used to deliver the intragastric members 218 into the main body 215 wherein the intragastric members 218 are stabilized within the intragastric balloon 211 by the foam material 212. The intragastric members 218 may be configured to provide dimensional stability to the intragastric balloon 211, or to reduce the weight or mass of the intragastric balloon 211.

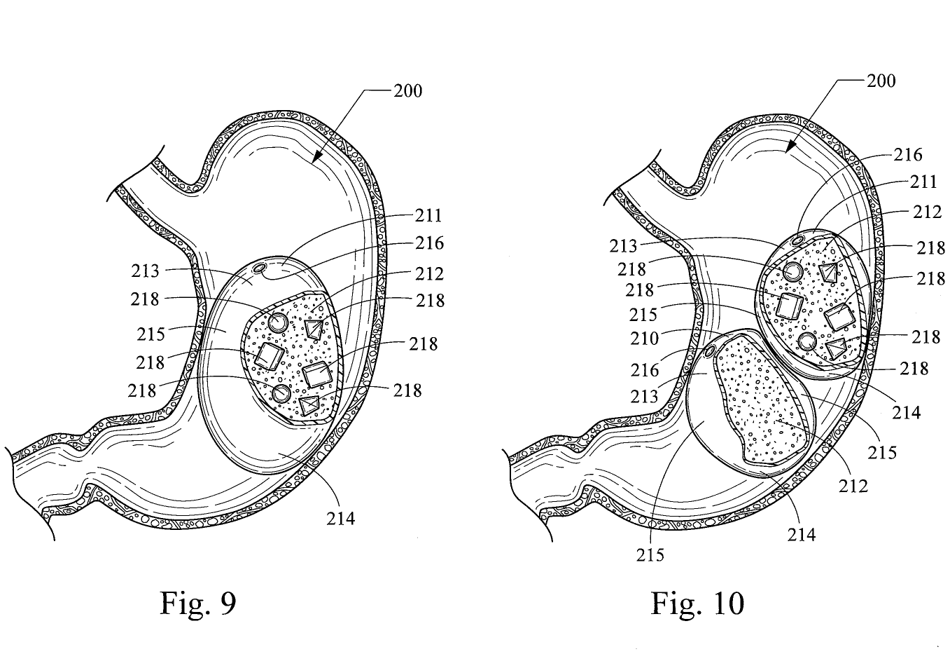

Upon receiving foam material 212, the intragastric balloon 211 is expanded from the first configuration to a second configuration, wherein the intragastric balloon 211 is sufficiently large to prevent the intragastric balloon 211 from passing through the mammal's pylorus (see FIG. 9). The expanded intragastric balloon 211 comprising the foam material 212 and the intragastric members 218 disposed within the foam material 212 engages the wall of the gastric lumen and reduces the volume of the gastric lumen thereby providing a feeling of fullness.

FIG. 10 depicts yet another embodiment of the obesity treatment apparatus 200 of FIG. 9 comprising a plurality of foam filled intragastric balloons 210, 211. In this embodiment, the first intragastric balloon 210 comprises foam material 212 and the second intragastric balloon 211 comprises foam material 212 and multiple intragastric members 218 disposed within the foam material 212. The intragastric balloons 210, 211 each comprise a preformed expandable digestive-resistant material having a spherical shape. However, in alternate embodiments of the present invention, the obesity apparatus 10 may include varying numbers of intragastric balloons, such as three or four, as well as various shapes and sizes. Additionally, the intragastric balloons 210, 211 may include varying numbers of intragastric members 218 disposed in varying amounts of foam material 212.

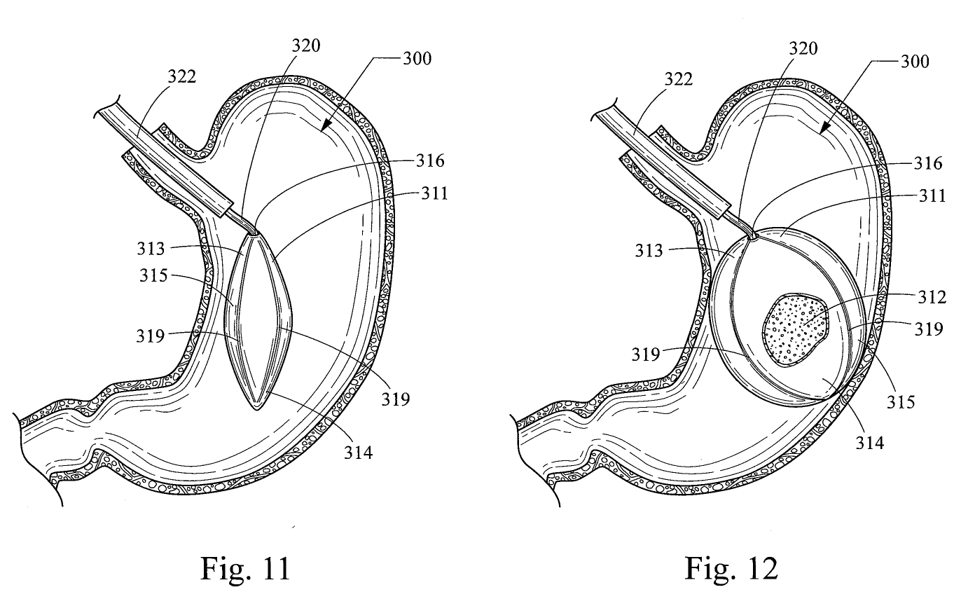

FIGS. 11-13 depicts another embodiment of the obesity treatment apparatus 300 comprising a single intragastric balloon 311 comprising foam material 312 and at least one reinforcement member 319. The reinforcement member 319 comprises expandable ribs aligned longitudinally that bow outwardly upon delivery to expand the intragastric balloon 311 in the gastric lumen of a mammal. The reinforcement member 319 further comprises nitinol or similar material. The intragastric balloon 311 comprises a preformed expandable digestive-resistant material having a spherical shape. The intragastric balloon 311 includes a proximal end 313, a distal end 314 and a main body 315, wherein the proximal end 313 provides an opening 316 to receive an inflation tube 320.

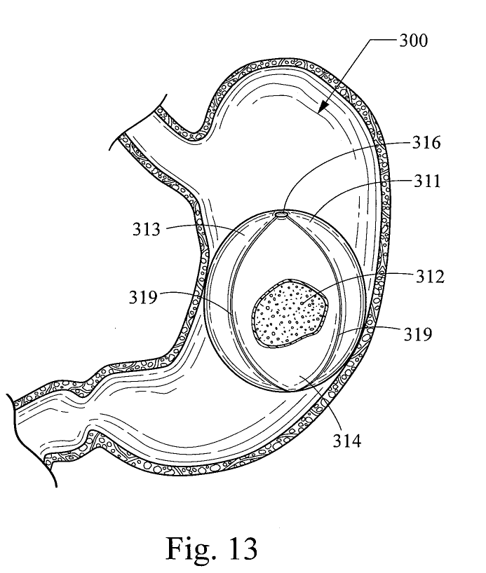

The intragastric balloon 311 is delivered into the gastric lumen of a mammal in a first configuration, wherein the first configuration is configured to permit introduction of the intragastric balloon 311 (see FIG. 11). The expandable ribs of the reinforcement member 319 expand upon delivery of the intragastric balloon 311 to the gastric lumen. The inflation tube 320 is then passed through an outer tube 322 and attached to the intragastric balloon 311 to insert foam material 312 into the main body 315 of the intragastric balloon 311 (see FIG. 12). Upon receiving foam material 312, the intragastric balloon 311 is further expanded to a second fully expanded configuration, wherein the intragastric balloon 311 is sufficiently large to prevent the intragastric balloon 311 from passing through the mammal's pylorus (see FIG. 13). The expanded intragastric balloon 311 engages the wall of the gastric lumen and reduces the volume of the gastric lumen thereby providing a feeling of fullness.

As illustrated in FIGS. 1-13, varying shapes are desired to increase the amount of volume or space occupied by the corresponding intragastric balloon comprising foam material. Particularly, the varying shapes can provide a feeling of fullness upon engaging the gastric lumen of the patient, i.e., the stomach walls of the patient. Additionally, the intragastric balloon may comprise intragastric members disposed within the foam material having varying shapes and designs that engage each other to displace volume after placement into the intragastric balloon (see, e.g., FIGS. 9-10) within the gastric lumen of the patient. Additionally, the intragastric members may include suitable materials such as synthetic organic polymers, polyurethanes, polyesters, carboxylated butadiene-styrene rubbers, and polyacrylates or other suitable material. The intragastric members are not limited to one particular shape, but can comprise varying shapes depending on the particular use. The shapes of the constituent components can be selected from the group consisting of circular, round, elliptical, square, triangular, rectangular, pentagonal, hexagonal, or any other suitable three dimensional shape. It should be appreciated that other designs utilizing expandable or alterable shapes could also be utilized. For example, intragastric members can be inflated or injected with foam material and subsequently disposed within the intragastric balloon.

The foam material of the present invention may include suitable materials such as synthetic organic polymers, polyurethanes, polyesters, carboxylated butadiene-styrene rubbers, polysiloxanes, polyacrylates, and sol-gels. The foam material may comprise polymeric foams including one or more types of monomers (e.g., copolymers) or mixtures (e.g., blends) of polymers. Other suitable foam materials may also include thermoplastic polymers (e.g., those that soften when exposed to heat and return to their original condition when cooled). Preferred foam materials of the present invention include sol-gels and multi-component polysiloxane mixtures which are mixed to cure to a foam-like structure, as will be explained below.

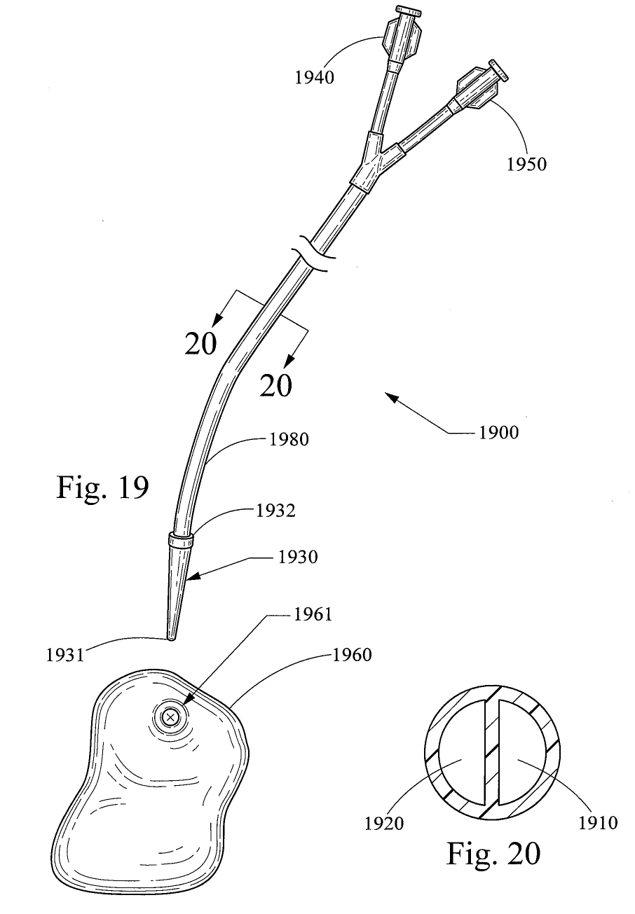

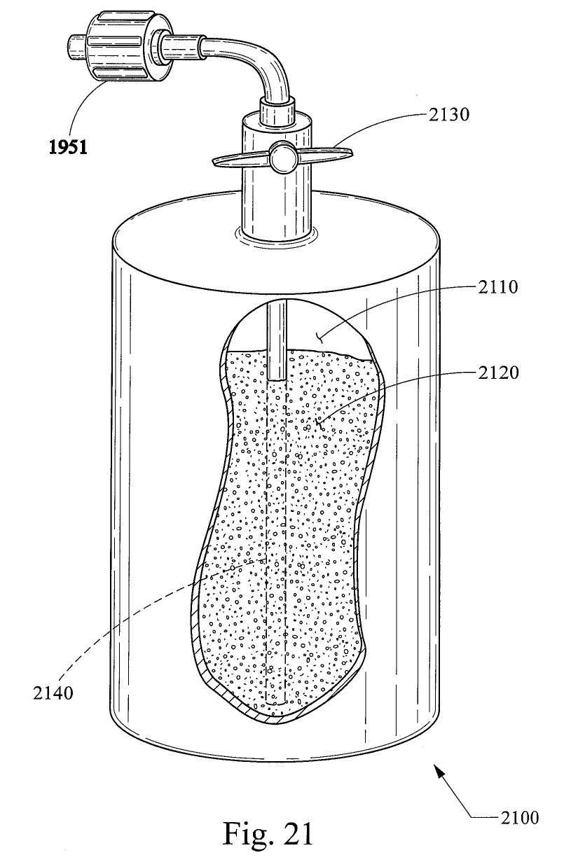

The foam material may be synthesized in numerous ways. In one exemplary method, foam components include an organosiloxane and a catalyst. The organosiloxane may be a poly(dimethylsiloxane) and the catalyst may be a tin compound, such as stannous octanoate. The poly(dimethylsiloxane) is cured to a foam-like structure within the intragastric balloon 1960 (i.e., gastric bag) at body temperature in the presence of the stannous octanoate catalyst. A multi-lumen catheter 1900 may be mechanically connected to canisters which hold liquefied phases of the catalyst and polysiloxane under pressure. Specifically, luer connector 1940 connects to a corresponding canister (not shown) housing the catalyst (FIG. 19). Luer connector 1950 of multi-lumen catheter 1900 connects to a corresponding luer connector 1951 of another canister 2100 (FIGS. 19 and 21) housing the poly(dimethylsiloxane). The poly(dimethylsiloxane) and the catalyst are preferably contained in a liquid flowable phase within each of their respective canisters. Each of the materials is introduced into their respective dedicated lumens 1910 and 1920 (FIG. 20) from their respective canisters.

Compressed gas or compressed air 2110 is disposed above the liquid precursor polymeric material 2120 to maintain the material 2120 under pressure. The compressed gas or air 2120 pushes the liquid material 2120 into feeding tube 2140. The compressed gas or air 2120 is maintained at a suitable pressure to enable the material to flow through catheter 1900 and into gastric bag 1960 when a valve 2110 located downstream of the canisters is opened. When the valve 2110 is opened from its closed position, at least a portion of the pressurized liquid poly(dimethylsiloxane) material 2120 flows upwards through the feeding tube 2140 and out from the canister 2100. Similarly, at least a portion of the catalyst (not shown) flows out from its respective canister. The poly(dimethylsiloxane) material 2110 and catalyst material emerge from their respective canisters and flow through separate respective lumens 1910 and 1920 of a multi-lumen catheter 1900 (FIG. 19). The lumens 1910 and 1920 ensure that the desired curing reactions do not take place until the materials enter the gastric bag 1960. Upon entering the gastric bag 1960, the materials within less than about 2 minutes cure to a foam-like structure. When the materials emerge from a distal end 1931 of catheter 1900 and enter the interior of the gastric bag 1960, the catalyst induces curing of the poly(dimethylsiloxane) at body temperature so as to increase in volume while simultaneously solidifying. The foam-like structure involves liberation of hydrogen gas, which is preferably removed from the bag 1960 by suctioning through a lumen of the catheter 1900. A one-way valve 1961 disposed along the bag 1960 secures the contents within the intragastric balloon 1960. The final cured elastomeric foam may possess a sufficient volume so as to not pass through the pylorus (FIG. 3).

An optional foam stabilizing agent (e.g., fluorinated silicones) may be used to enhance the foaming phenomenon that occurs during curing of the poly(dimethylsiloxane). The foam stabilizing agents may be introduced into the gastric bag 1960 (prior to, during, or after introduction of the catalyst and polyorganosiloxane into bag 1960) to facilitate curing when the materials have not been mixed within catheter 1900 prior to entering the gastric bag 1960.

Although the two components (i.e., poly(dimethylsiloxane) and stannous octanoate catalyst) have been described as not requiring mixing prior to entering the intragastric balloon 1960, the multi-lumen catheter 1900 may comprise a mixing region, such as a baffled static mixer 1930 at the distal end 1931, to mix the components as shown in FIG. 19. The materials exit their respective lumens 1910 and 1920 (FIG. 20) along the distal end 1932 of shaft 1980 and thereafter enter into the static baffled structure 1930. The baffles 1930 may create turbulent eddy flow so as to sufficiently contact and mix the materials therewithin baffled structure 1930 to promote the onset of curing. The materials preferably exit the distal end 1931 of the baffled structure 1930 after the onset of curing but prior to complete curing, which may occur within about 2 minutes or less. The partially cured material emerges from the distal end 1932 of baffled structure 1930 and enters the gastric bag 1960. As the materials continue to cure and solidify, gas (e.g., hydrogen gas) is evolved as a by-product of the reaction which causes the polymerized material to expand and form the foam-like structure. Such off-gassing during formation of the foam is preferably suctioned off by applying vacuum to the proximal end of the catheter 1900 as known to one of ordinary skill in the art. The resultant elastomeric foam is chemically inert and occupies a volume within the gastric bag 1960 sufficient to engage one or more walls of the lumen to induce satiety.

The intragastric balloon 1960 may be a pliable gastric bag capable of withstanding the initial pressures produced during polymerization and expansion of the foam material. The gastric bag 1960 may be deployed within the gastric lumen as described by any of the delivery procedures described. The bag 1960 may be anchored to one or more walls of the gastric lumen as known in the art.

The effective amounts of each of the materials necessary to induce curing to create the foam structure may depend upon numerous factors, including the size of the balloon 1960. The poly(dimethylsiloxane) and catalyst are preferably mixed in a sufficient weight ratio so that the catalyst induces curing of all of the poly(dimethylsiloxane).

Other suitable polysiloxane precursors are contemplated. For example, a 2-component polyorganosiloxane mixture is contemplated such that the mixing of the two components at predetermined proportions and at body temperature facilitates curing and foaming. The first component may be a hydroxy polysiloxane. The second component may be an alklylhydrogensiloxane. A platinum catalyst may be used to promote the reaction between the first and the second components. By virtue of the plurality of silicon-bonded hydrogen atoms and silicon bonded hydroxyl groups of the polysiloxanes, a network of cross-linked interconnected polysiloxane chains is produced and hydrogen gas is evolved and thereafter suctioned from the bag 1960. The liberated hydrogen gas assists in formation of the expanded foam-like structure. The composition cures to form long chain polymer molecules having the repeating unit (R—Si—O—Si) in which R represents a hydrocarbon chain or phenyl group. Because cross-linking creates interconnected branching of the polymer chains, a foam structure with sufficient volume may be created within the gastric bag 1960. Delivery of the 2-component siloxane is achieved by delivering the first component in lumen 1920 and the second component in lumen 1910, and the catalyst in a separate third lumen (not shown). The optional baffled structure 1930 may be used to facilitate mixing and onset of curing prior to entry into the bag 1960. Alternatively, the first and the second components 1910 and 1920 may directly enter the bag 1960 without any prior mixing.

The final density of the foam structure within the gastric bag 1960 may be altered by introducing one or more suitable alcohols (e.g., ethanol, n-propyl alcohol), preferably after the start of the curing between the first and the second components within gastric bag 1960. The one or more alcohols may contribute to hydrogen gas generation and cross-linking between polymer molecules so as to enhance expansion of the polymerized mixture into the resultant foam-like structure. Other materials having functional hydroxyl groups may also be used to vary the final density to the desired value.

Another example of silicone foam components includes a first component of a polydiorganosiloxane having not less than three alkylhydrogensiloxane units and a second component of a polydiorganosiloxane having not less than two siloxane units. The first and the second components are introduced through lumens 1910 and 1920 along with a suitable catalyst (e.g., platinum-based catalyst) in a third lumen (not shown). Separation of the components within respective lumens 1910 and 1920 prevents premature curing from occurring until the components enter into the gastric bag 1960. After curing has proceeded within the bag 1960, a liquid alcohol to facilitate cross-linking of the polymer molecules and a fluorinated silicon foam stabilizing agent to enhance foaming may be introduced into the gastric bag 1960.

Although silicone and siloxane precursor materials have been included, any non-toxic biocompatible chemical precursor capable of curing from a flowable liquid phase to a foam-like structure within the gastric bag 1960 at body temperature is contemplated.

Other suitable materials may be used which do not require delivery within a multi-lumen catheter 1900. For example, biocompatible sol-gels which transition from a liquid phase into a gel phase at body temperature may be utilized to form the foam. The sol-gel is a liquid at ambient temperature and gels to a thickened, non-flowing viscous consistency at human body temperature. In one example, aqueous solutions of polyoxyethylene-polyoxypropylene (POE-POP) may be used. A predetermined weight percentage of the POE-POP sol-gel is preferably dissolved in purified (e.g., distilled, filtrated, ion-exchanged) water. Preferably, the weight percentage of POE-POP ranges from about 17% to about 26% with the balance being water. The POE-POP may be introduced as a liquid at ambient temperature from its storage canister through a single lumen catheter. A mixing structure (e.g., baffled structure 1930) at the distal portion of the catheter is not necessary. When the POE-POP sol-gel enters the gastric bag 1960, it begins to cure because it possesses a gel transition temperature at human body temperature. The predetermined weight percentage of the POE-PDP is selected so as to enable the sol-gel to transition into a gel at body temperature. The gel transition temperature may be raised or lowered by varying the weight percentage of the POE-POP. Generally speaking, the higher the weight percentage of the POE-POP polymer, the lower the gel-transition temperature.

Other sol-gel materials may be used, which tend to undergo a liquid-gel phase transition which gels in situ under the effect of an increase in its ionic strength. As an example, an aqueous solution containing a predetermined weight percentage of a suitable ionic polysaccharide (e.g., gellan gum) is liquid at low ionic strength but undergoes a liquid phase to gel phase transition when the ionic strength is increased by addition of a cationic gelling agent, such as calcium, for example, in the form of a divalent or trivalent cation. For example, a suitable polysaccharide may be introduced into the gastric bag 1960 and thereafter a suitable salt (e.g., sodium chloride, potassium chloride, sodium sulfate) having a predetermined molar strength may be injected into the bag 1960 so as to increase the ionic strength of the polysaccharide to a threshold level where the gel-transition temperature decreases to induce gelling within the bag 1960. Other suitable gel forming ionic polysaccharides include alginate gums and chitosan. Still other suitable gel forming materials include polyols.

Varying the pH of the deployed sol-gel may also be an additional and/or alternative means to control the gel-transition temperature. Generally speaking, lowering the pH will increase the gel-transition temperature of the sol-gel material. The pH may be adjusted by adding an appropriate amount of a biological acid or base known to those skilled in the art, such as hydrochloric acid or sodium hydroxide.

Suitable foam material of the present invention may include a wide range of thicknesses. Furthermore, the foam material can include one or more layers directly bonded to each other or bonded together with adhesive and/or tie layers, as long as the overall properties of the foam material, as described herein, are biocompatible with the gastric lumen of the mammal. Optionally, disposed between these layers can be one or more layers of polymeric netting or nonwoven, woven, or knit webs for enhancing the physical integrity of the foam material.

The foam material may be hydrophilic, hydrophobic or may be treated with surfactants, such as nonionic surfactants, to render them more hydrophilic. In the embodiment of the present invention comprising both foam material and intragastric members disposed within the foam material (see, e.g., FIGS. 9-10), the foam material may be coextensive with the intragastric members in the main body of the intragastric balloon. For these embodiments, the foam material can also include an adhesive to bond the periphery of the foam material to a surface of the intragastric members or the main body of the intragastric balloon.

Deployment of the intragastric balloons of the present invention can be accomplished in a number of ways, depending on the size, number, and configuration of the devices, or according to physician or patient preference.

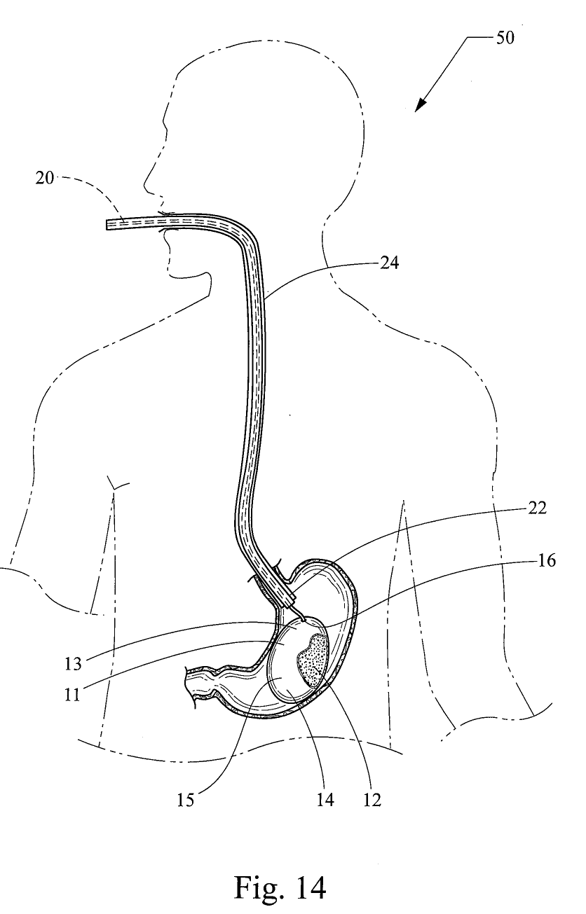

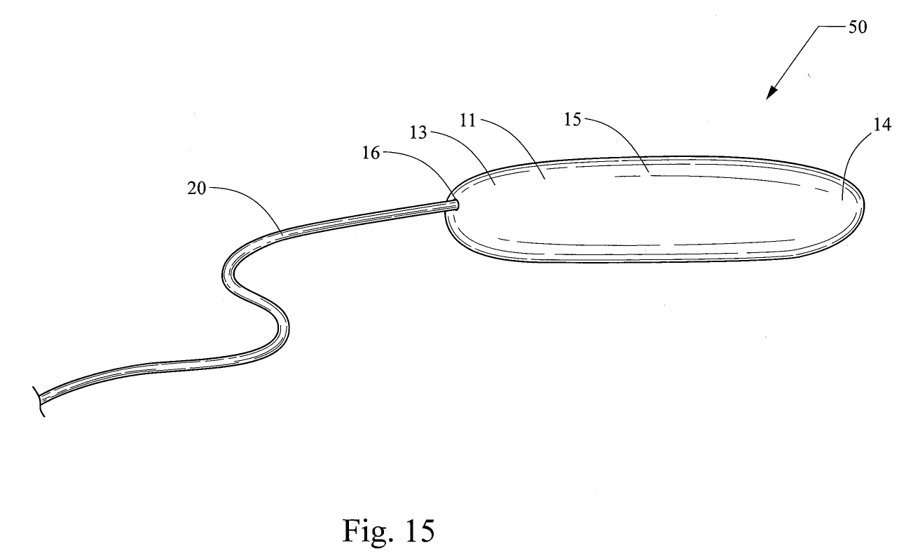

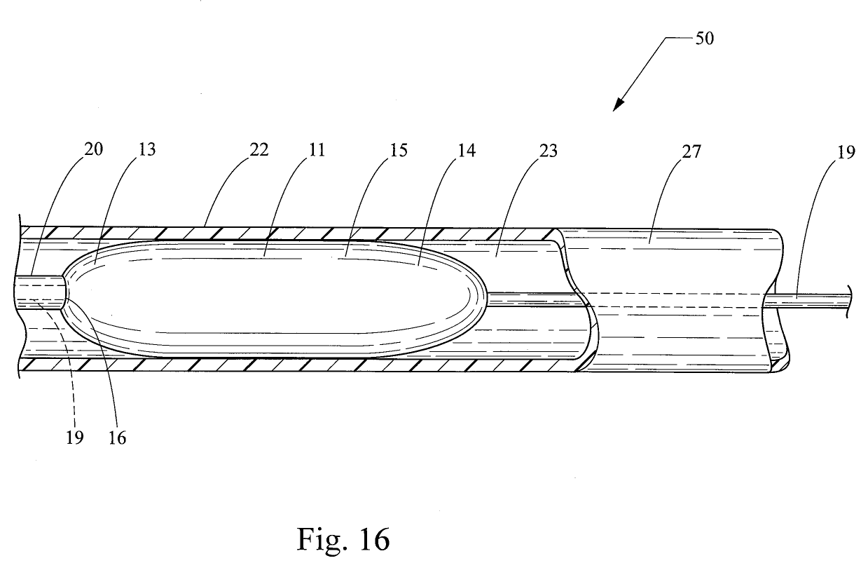

FIGS. 14-16 depict one such delivery system 50 in which at least one intragastric balloon 11 is delivered into the gastric lumen within outer tube 22, such as a sheath, tube, package, wrapping, etc., and subsequently released. For example, the intragastric balloon 11 (or multiple balloons) is preloaded into outer tube 22 or introducer, then deployed therefrom by being pushed out by using a pusher member (not shown). As shown in FIGS. 15-16, the intragastric balloon 11 is shown in a first configuration, wherein the intragastric balloon 11 is compressed to aid in loading within outer tube 22 for delivery. Subsequently, the intragastric balloon 11 is deployed into the gastric lumen (see FIG. 14). A wire guide 19 is typically used in the procedure, and is placed through a passageway 23 of the outer tube 22.

FIG. 14 depicts an overtube 24 that is used to deliver the intragastric balloon 11 into the gastric lumen of the patient. The overtube 24 is used in combination with an endoscope to establish a passageway to a target delivery site in the stomach. Once the overtube 24 is positioned in the gastric lumen of the patient, the distal end 14 of the intragastric balloon 11 is passed through the outer tube 22 until the intragastric balloon 11 reaches the gastric lumen. Once the proximal end 13 of the intragastric balloon 11 is delivered into the gastric lumen, foam material 12 is delivered into the main body 15 of the intragastric balloon 11 through the inflation tube 20, thereby expanding the intragastric balloon 11 to a second configuration.

After the intragastric balloon 11 has been inflated with foam material 12 to its intended volume, the inflation tube 20 is disengaged from the intragastric balloon 11. The inflation tube 20 may then be removed from the gastric lumen wherein the intragastric balloon 11 remains within the gastric lumen of the patient.

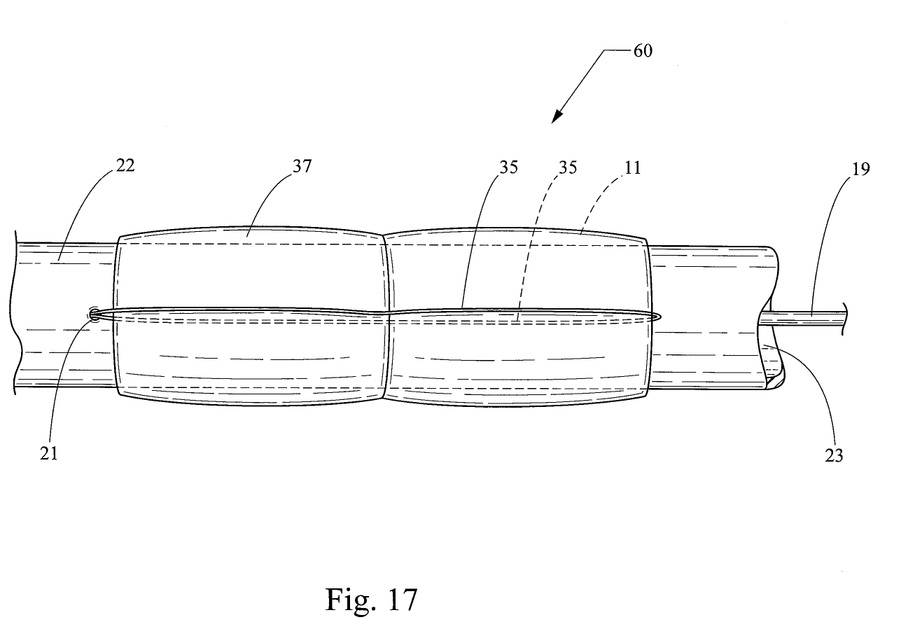

FIG. 17 depicts a delivery system 60 in which the intragastric balloon 11 is loaded over the outer tube 22 (as in FIG. 15), but is secured by wrapping a splittable sheath 37 or sleeve made of a thin plastic material around the intragastric balloon 11. In the illustrative embodiment, a releasing mechanism 35 comprises a nylon thread or wire that is looped under and over the sheath 37, such that it can be withdrawn to tear through the thin material of the sheath 37 to release the intragastric balloon(s) 11 mounted on the outer tube 22. The releasing mechanism 35 feeds into an aperture 21 and passageway 23 of the overtube 22, where it extends to the proximal end of the intragastric balloon 11. Other types of splittable sheaths 37 can also be used, such as the COOK® PEEL-AWAY Introducer Sheath.

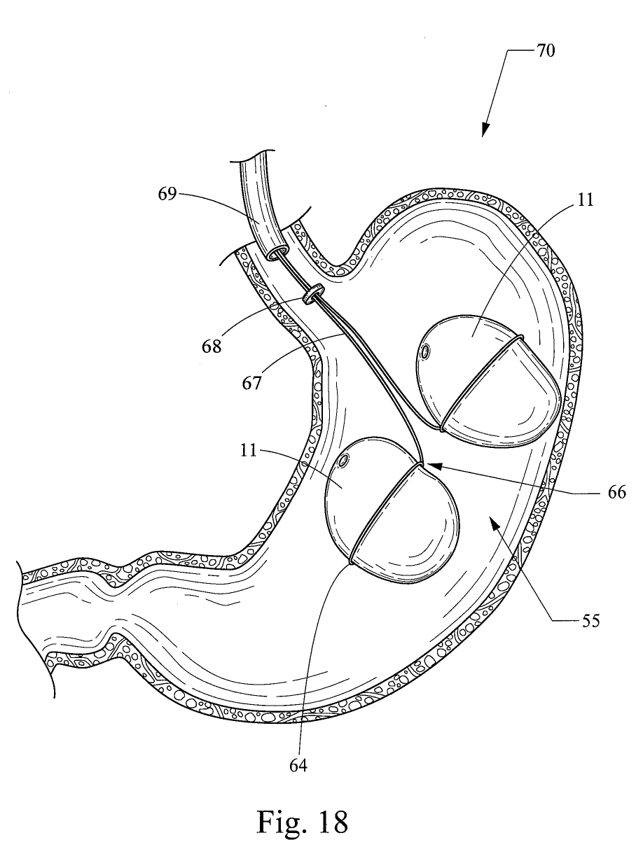

FIG. 18 depicts a delivery system 70 providing a plurality of intragastric balloons 11 delivered into the gastric lumen of the patient. In this embodiment, it may be necessary that the intragastric balloons 11 be coupled together to form a grouping or set 55 of intragastric balloons 11 to retain the intragastric balloons 11 within the gastric lumen. The two deployed intragastric balloons 11 each have a coupling mechanism 66 (tether 67) attached about them such that they can be drawn together as depicted in FIG. 18. A push member 69, such as a corrugated metal tube, is placed into gastric lumen by using an endoscope, and is guided over the tethers 67 to urge a securing element 68, such as a rubber patch, tightly against the two intragastric balloons 11. The tethers 67 can then be cut, allowing the grouping 55 to float free within the stomach. This method can also be used to join additional intragastric balloons 11 to form a larger grouping 55.

Likewise, the illustrative delivery system 50 of FIGS. 14-16 can be used to deliver any practical number of intragastric balloons 11, which can then be joined in the manner described above, or they can be delivered singly or in pairs, and then grouped together after all of the intragastric balloons 11 have been placed. The adherence of mucous and other changes that occur within the gastric lumen environment can, over time, significantly increase the volume of the intragastric balloons 11. The increased size can make it very difficult to remove the grouping from the stomach. To address this problem, multiple intragastric members 11, are grouped together after introduction into the gastric lumen and then cut apart when it is time to remove them from the patient.

Additionally, the illustrative embodiments of delivery systems of the present invention can also be utilized to deliver intragastric members 218 (see FIGS. 8-10) into the foam material 212 of the intragastric balloon 211 in a number of ways, depending on the size, number, and configuration of the intragastric balloon 211, or according to the physician's preference. Likewise, the intragastric members 218 can be joined together, or they can be delivered singly or in pairs, and grouped together after all the intragastric members 218 have been disposed within the foam material 212 in the main body 215 of the intragastric balloon 211. In addition, this embodiment of the intragastric balloon 211 can be delivered into the gastric lumen of the patient in a number of ways, including the manner as described above.

For example, one delivery system utilizes an elastic band (not shown) attached to the opening of the intragastric balloon 211 which is inserted over an overtube wherein the remainder of the balloon 211 is inverted into the lumen of the overtube. As illustrated in the embodiment depicted in FIG. 8, upon delivery into the intragastric balloon 211, the intragastric members 218 are subsequently pushed into the intragastric balloon 211 and disposed with any foam material 212 until the intragastric balloon 211 is filled at a predetermined volume. Additionally, a coaxial outer tube or similar device can be utilized to remove the elastic band from the overtube and thereby secure the intragastric balloon 211 with the elastic band. The elastic band is configured to elastically retract around the opening of the intragastric balloon 211 after being removed from the overtube to secure the intragastric members 218 within the foam material 212 of the intragastric balloon 211. This delivery system can be utilized to deliver intragastric members 218 of various configurations and may include intragastric members 218 that are preloaded onto a delivery tube. In another embodiment, trigger wires or the like can be connected proximal to the overtube, wherein the trigger wires are used to expel the elastic band from the overtube.

The intragastric balloon 211 and the disposed foam material 212 may be removed by rupturing the intragastric balloon 211, resulting in the foam material 212 and other intragastric matter, such as intragastric members 218, being released from the intragastric balloon 211 and passing through the gastrointestinal tract of the patient. Further, the foam material 212 can include a color coding to allow the foam material 212 to be easily identified if the intragastric balloon 211 is prematurely ruptured. For example, the color coded foam material 212 can provide notification to the physician or patient when identified in stool samples. In an alternate embodiment, the foam material 212 can be removed by rupturing the intragastric balloon 211 and utilizing an overtube to suction the foam material 212 from the intragastric balloon 211 and subsequently removing the intragastric balloon 211 through the overtube or endoscope with forceps or similar device.

As illustrated in FIGS. 1-18, the intragastric balloon of the present invention may include any shape suitable to receive foam material and thereby increasing the amount of volume or space occupied within the gastric lumen. Particularly, the structure and shape of the intragastric balloon includes any shape that provides a feeling of fullness upon engaging the stomach walls of the patient, such as an oval, circle, triangle, square and rectangle. The varying shapes of the intragastric balloon further provide complimentary designs to properly receive the varying shapes of the intragastric member after placement into the intragastric balloon. The intragastric balloon may also include an inner member to seal the inner reservoir of the intragastric balloon after delivery of foam material into the intragastric balloon.

In the embodiments illustrated, the intragastric balloon can comprise a preformed expandable digestive-resistant material, such as latex, elastic, or any other suitable material. Other suitable materials include polytetrafluoroethylene (PTFE), polyethylene terephthalate, polyester, polyurethane, silicone, Dacron, Thoralon, polypropylene knit, and other material which will be apparent to those of skill in the art in view of the present invention. Alternatively, the intragastric balloon can comprise degradable materials having coatings comprising indigestible polymers and the like. The intragastric balloon is not limited to the above designs and can include alternative embodiments consisting of gastric socks, pouches or similar devices. Thus, the intragastric balloon is available in a variety of materials, sizes, shapes and diameters, which result in varying designs and configurations during advancement and placement within the gastric lumen.

The intragastric balloon may also comprise either a resilient elastomeric material or a substantially non-compliant material. An intragastric balloon comprising resilient elastomeric material provides the ability to stretch when filled with the intragastric members. Conversely, intragastric balloons comprising substantially non-compliant material provides the ability to form a predetermined final shape and volume when filled with foam material.

A method of treatment of obesity in mammals can comprise the steps of positioning one or more intragastric balloons within the gastric lumen of a mammal and delivering a foam material into the intragastric balloon, wherein the intragastric balloon is expanded from a first configuration to a second configuration upon delivery of the foam material into the one or more intragastric balloons. The second configuration is sufficiently large to prevent the one or more intragastric balloons comprising foam material from passing through the mammal's pylorus. The method also comprises the additional step of advancing the foam material through a lumen of an inflation tube attached to the one or more intragastric balloons within the gastric lumen.

The method can also comprise the step of positioning a delivery tube comprising the one or more intragastric balloons within a lumen of an overtube. The method further comprises the step of advancing an intragastric member through the lumen of the overtube and disposing the intragastric member within the foam material of the intragastric balloon. In addition, the method further comprises the step of securing a stopper to an opening of the one or more intragastric balloons within the gastric lumen.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention.

1. An intragastric device for the treatment of obesity, the intragastric device comprising:
one or more intragastric balloons disposed within the gastric lumen of a mammal; and
a biocompatible foam material disposed within the one or more intragastric balloons, the foam material being configured to permit introduction of the foam material into the one or more intragastric balloons, wherein when the foam material is disposed within the one or more balloons, the one or more intragastric balloons are configured to prevent the intragastric device from passing through the mammal's pylorus.

2. The intragastric device according to claim 1, wherein the foam material comprises an organosiloxane.

3. The intragastric device according to claim 2, wherein the organosiloxane is a poly(dimethylsiloxane).

4. The intragastric device according to claim 3, wherein dimethylsiloxane is adapted to be polymerized by a catalyst and thereafter cure at body temperature to form the poly(dimethylsiloxane).

5. The intragastric device according to claim 2, wherein the organosiloxane comprises a first component and a second component.

6. The intragastric device according to claim 5, wherein the first component comprises polysiloxane having one or more hydroxyl functional groups and the second component comprise a polysiloxane having one or more alkyl hydrogen groups.

7. The intragastric device according to claim 1, wherein the foam comprises a sol-gel.

8. The intragastric device according to claim 7, wherein the sol-gel comprises a co-polymer of polyoxyethylene and polyoxypropylene, the co-polymer adapted to transition from a liquid phase to a gel phase within the one or more intragastric balloons disposed within the gastric lumen.

9. The intragastric device according to claim 1 wherein an inflation tube is attached to an opening of the one or more intragastric balloons for delivering foam material into the one or more intragastric balloons.

10. The intragastric device according to claim 1 wherein the one or more intragastric balloons are expandable from a first configuration to a second configuration upon receiving the foam material, wherein the second configuration is sufficiently large to prevent the one or more intragastric balloons from passing through the mammal's pylorus.

11. The intragastric device according to claim 1 wherein the one or more intragastric balloons are connected together with a releasing mechanism that passes through an opening in the one or more intragastric balloons upon delivery into the gastric lumen.

12. The intragastric device according to claim 11 wherein the releasing mechanism comprises a nylon thread having a first end and a second end that are connected together for securing the one or more intragastric balloons upon delivery into the gastric lumen, and that is released upon removal of the one or more intragastric balloons from within the gastric lumen.

13. The intragastric member of claim 1 wherein the one or more intragastric balloons are loaded through a delivery tube, wherein the delivery tube facilitates the delivery of the one or more intragastric balloons into the gastric lumen.

14. The intragastric device according to claim 1 further comprising an overtube comprising a proximal end, a distal end and a lumen configured to receive the one or more intragastric balloons.

15. The intragastric device according to claim 1 wherein the one or more intragastric balloons comprise a reinforcement member comprising nitinol allowing the one or more intragastric balloons to expand from a first configuration to a second configuration.

16. A method of treatment of obesity in mammals, the method comprising the steps of:
positioning one or more intragastric balloons within the gastric lumen of a mammal; and
delivering a foam material into the one or more intragastric balloons, wherein the one or more intragastric balloons are expanded from a first configuration to a second configuration upon delivery of the foam material into the one or more intragastric balloons, wherein the second configuration is sufficiently large to prevent the one or more intragastric balloons from passing through the mammal's pylorus.

17. The method of claim 16, wherein the step of delivering the foam material comprises the steps of:
providing a catalyst canister comprising a catalyst stored under pressure;
providing a precursor foam canister comprising a precursor foam material stored under pressure;
introducing the catalyst through a first lumen of a multi-lumen catheter;
introducing the precursor foam material through a second lumen of the multi-lumen catheter;
advancing the catalyst and the precursor foam material beyond a distal end of the multi-lumen catheter and into the or more intragastric balloons within the gastric lumen;
polymerizing at body temperature the precursor foam material within the one or more balloons in the presence of the catalyst; and
expanding the precursor material to a volume sufficient to engage one or more walls of the gastric lumen; and
curing the precursor foam material into the foam material.

18. The method of claim 17, wherein the distal end of the multi-lumen catheter comprises a baffled static mixer into which the catalyst and the precursor foam material enter and mix prior to entering the one or more intragastric balloons within the gastric lumen.

19. The method of claim 17, wherein the step of expanding in volume the precursor material further comprises liberating gas that is removed from the one or more intragastric balloons by suctioning.

20. The method of claim 17, further comprising the step of introducing a biocompatible foaming agent into the balloon.

21. The method of claim 17, further comprising the step of introducing a biocompatible alcohol into the one or more balloons to enhance expansion of the precursor material during polymerization.

22. The method of claim 16, further comprising the steps of:
providing a sol-gel canister comprising a biocompatible sol-gel stored as a liquid phase under pressure within the canister;
introducing the sol-gel in the liquid phase from the canister into a lumen of a catheter;
advancing the sol-gel beyond a distal end of the catheter and into the or more intragastric balloons within the gastric lumen; and
gelling the liquid phase sol-gel to a gel phase within the one or more balloons.

23. The method of claim 22, wherein the sol-gel has a liquid-to-gel transition temperature at a body temperature of the mammal.

24. The method of claim 22, wherein the sol-gel has a liquid-to-gel transition temperature at a predetermined ionic strength or pH.

25. The method of claim 22, wherein the sol-gel has a composition comprising a polyoxyethylene-polyoxypropylene co-polymer.
